(19) United States
(12) Patent Application Publication
(10) Pub. No.: US 2008/0280854 A1
(43) Pub. Date: Nov. 13, 2008

(54) PREVENTING OR REDUCING OXIDATIVE STRESS OR OXIDATIVE CELL INJURY

(76) Inventors: Wallace H. Yokoyama, Berkeley, CA (US); Qiming Shao, Fremont, CA (US); Maciej Turowski, Midland, MI (US); Stephanie K. Lynch, Zurich (CH)

Correspondence Address: The Dow Chemical Company, Intellectual Property Section, P.O. Box 1967, Midland, MI, 48641-1967, US

(21) Appl. No.: 12/051,249

(22) Filed: Mar. 19, 2008

Related U.S. Application Data
(62) Division of application No. 11/289,914, filed on Nov. 30, 2005, now abandoned.

(51) Int. Cl. A61K 31/717; A61P 43/00

(52) U.S. Cl. 514/57

(57) ABSTRACT

A water-soluble cellulose derivative is useful for preventing or reducing oxidative stress or oxidative cell injury in tissues of an animal and in particular for regulating Stearoyl-CoA Desaturase-1 gene expression and/or ATPF1 gene expression in non-adipose tissues of the animal.

This application is a divisional (continuation) of copending application Ser. No. 11/289,914 filed Nov. 30, 2005.

This invention was made under a Cooperative Research And Development Agreement with the U.S. Department of Agriculture, number 58-3K95-5-1072.

### FIELD OF THE INVENTION

This invention relates to the prevention or reduction of oxidative stress or oxidative cell injury in tissues of an animal.

### BACKGROUND OF THE INVENTION

Oxidative stress is generally defined as an excess production of oxidizing agents in tissues. It is generally accepted in the medical sciences that oxidative stress can lead to cell injuries and eventually to cell death in such tissues.

Under normal physiological conditions, the use of oxygen by cells of aerobic organisms generates potentially deleterious reactive oxygen metabolites. A chronic state of oxidative stress exists in cells with an imbalance between prooxidants/oxidants and antioxidants. The amount of oxidative damage increases as an organism ages and is postulated to be a major causal factor of senescence (R S Sohal and R. Weindruck, Department of Biological Sciences, Southern Methodist University, Dallas, Tex. 75275, USA. Science, 1996 July 5; 273(5271):59-63).

In view of the great importance of preventing or reducing oxidative stress or oxidative cell injury in tissues of animals, particularly of human beings, tremendous effort has been spent on finding medicinal antioxidants. As disclosed in U.S. Pat. No. 6,204,295, medicinal antioxidants are compounds that may be used for the prevention of tissue damage induced by lipid peroxidation (Haliwell, B., FASEB J. 1:358-364, 1987). U.S. Pat. No. 6,204,295 discloses that during lipid peroxidation free radicals interact with polyunsaturated fatty acids to form lipid peroxyl radicals, which produce lipid hydroperoxides and further lipid peroxyl radicals. This peroxidative cascade may eventually consume an essential part of the membrane lipid of a cell, which may lead to changes in membrane permeability and ultimately in cell death.

Over the past decade substantial scientific evidence in a wide variety of biomedical fields has implicated oxidative-free-radical injury and, in particular, excess production of reactive oxygen species (ROS), as primary factors causing cell death and tissue injury in a number of clinically important diseases, including central nervous system degenerative diseases, long-term complications of diabetes, atherosclerosis, ischemic cardiovascular diseases, as well as sun-induced skin damage and physical manifestations of aging.

For example, Alexander R W, Department of Medicine, Emory University School of Medicine, Atlanta, Ga., USA, “Transactions of the American Clinical and Climatological Association” (1998), 109 129-45 discloses that accumulating evidence provides a compelling case that one of the major pathophysiologic mechanisms involved in the pathogenesis of atherosclerosis is enhanced oxidative stress and that the most important manifestation of this altered redox state is the modulation of a set(s) of proinflammatory genes that are regulated directly or indirectly by reactive oxygen species. The author theorizes that hypercholesterolemia, hypertension, and age related to diabetes mellitus all activate similar redox-sensitive proinflammatory genes.

In view of the huge importance of preventing or reducing oxidative stress or oxidative cell injury in tissues of animals, particularly of human beings, it would be highly desirable to find new methods which are useful for preventing or reducing oxidative stress or oxidative cell injury.

Studies on the enzyme Stearoyl-CoA Desaturase-1 (SCD1) have suggested that SCD1 appears to be an important metabolic control point, and inhibition of its expression could benefit the treatment of obesity, diabetes and other metabolic diseases. Stearoyl-Coenzyme A (CoA) Desaturase is a central lipogenic enzyme catalyzing the synthesis of saturated acids, mainly palmitic acid and stearic acid, to monounsaturated fatty acids, mainly palmitoleate and oleate (J M Ntambi, M. Miyazaki, Department of Biochemistry and Nutritional Sciences, University of Wisconsin, Madison, USA: “Recent insights into Stearoyl-CoA Desaturase-1”, Curr Opin Lipidol. 2003 June; 14(3):255-61). J M Ntambi and M. Miyazaki disclose that mice that have a naturally occurring mutation in the SCD1 gene iso-form as well as a mouse model with a targeted disruption of the Stearoyl-CoA Desaturase gene-1 (SCD1−/−) have revealed the role of de-novo synthesized oleate and thus the physiological importance of SCD1 expression. It was found that SCD1−/− mice had reduced adiposity, increased insulin sensitivity, and are resistant to diet-induced obesity.

SCD1 transcript has been found to be expressed in liver, lung, kidney, brain, stomach, muscle, adipose tissue, and skin. Fluorescent in situ hybridization showed that SCD1 expression in skin is restricted to the sebaceous glands, more specifically to the region containing mostly undifferentiated sebocytes, the bottom of the sebaceous gland (Ntambi et al., 1995; Ntambi et al., 1988; Zheng et al., 1999; Zheng et al., 2001). Gene expression for ATP synthase, such as ATPAF1 (ATP synthase mitochondrial F1 complex assembly factor 1) gene expression, can also play an important role in preventing or reducing oxidative stress or oxidative cell injury in tissues of animals. ATP synthase is an enzyme that catalyzes the reaction of ATP synthesis and hydrolysis in the mitochondria. ATP (adenosine triphosphate) is used to provide energy for biochemical reactions, for example in the oxidation of fatty acids in the mitochondria in non-adipose tissues. Fatty acids are stored in the form of triacylglycerols primarily within adipocytes of adipose tissue. In response to energy demands, the fatty acids of stored triacylglycerols can be mobilized for use by non-adipose tissues. Fatty acids must be activated in the cytoplasm before being oxidized in the mitochondria. Activation is catalyzed by fatty acyl-CoA ligase (also called acyl-CoA synthetase or thiokinase). The net result of this activation process is the consumption of 2 molar equivalents of ATP.

Glucose and fatty acids are the ultimate sources of energy for animal cells. When glucose is scarce, fatty acids are mobilized for energy. A feature of insulin resistance is high concentrations of glucose and insulin in the blood, but a decreased transport of glucose into non-adipose tissues, such as peripheral tissues, despite high levels of insulin. Under these conditions fatty acids are converted to energy by mitochondria. While not wishing to be bound to the theory, Applicants believe that elevated gene expression for ATPAF1, a subunit of ATP synthase, is an indication of elevated oxidation of fatty acids in tissues, particularly in non-adipose tissues of animals, which can lead to oxidative stress or oxidative cell injury in such tissues.

In view of the substantial evidence that SCD1 is an important metabolic control point, it would be highly desirable to find a way of regulating the expression of one or more genes related to fat metabolism of tissues of an animal, preferably the expression of one or more genes inducing conversion of saturated fatty acids to monounsaturated fatty acids. It would be particularly desirable to find a way of reducing SCD1 gene expression in tissues of animals, particularly in non-adipose tissues. It would also be desirable to find a way of regulating the expression of one or more genes related to mitochondrial oxidation pathways, and in particular of regulating ATP synthase gene expression in tissues of animals, particularly in non-adipose tissues.

### SUMMARY OF THE INVENTION

It has surprisingly been found that administration of a water-soluble cellulose derivative is useful for reducing Stearoyl-CoA Desaturase-1 (SCD1) gene expression and for reducing ATP synthase mitochondrial F1 complex assembly factor 1 (ATPAF1) gene expression in tissues of animals.

One aspect of the present invention is a method of regulating the expression of a gene related to fat metabolism of tissues of an animal, which method comprises the step of administering to the animal an effective amount of a water-soluble cellulose derivative.

Another aspect of the present invention is a method of regulating, particularly reducing, Stearoyl-CoA Desaturase-1 gene expression in tissues of an animal, which method comprises the step of administering to the animal an effective amount of a water-soluble cellulose derivative.

Yet another aspect of the present invention is a method of regulating, particularly reducing, ATPAF1 gene expression in tissues of an animal, which method comprises the step of administering to the animal an effective amount of a water-soluble cellulose derivative.

Yet another aspect of the present invention is a method of preventing or treating a disease of an organ of an animal caused or facilitated by Stearoyl-CoA Desaturase-1 gene expression, particularly Stearoyl-CoA Desaturase-1 gene over-expression, which method comprises the step of administering to the animal an effective amount of a water-soluble cellulose derivative.

Yet another aspect of the present invention is a method of preventing or reducing oxidative stress or oxidative cell injury in tissues of an animal, which method comprises the step of administering to the animal an effective amount of a water-soluble cellulose derivative.

Yet another aspect of the present invention is a method of preventing or treating a disease of an organ of an animal caused or facilitated by oxidative stress or oxidative cell injury in said organ, which method comprises the step of administering to the animal an effective amount of a water-soluble cellulose derivative.

Yet another aspect of the present invention is a pharmaceutical composition, food or food supplement comprising an effective amount of a water-soluble cellulose derivative for regulating the expression of a gene related to fat metabolism of tissues of an animal.

Yet another aspect of the present invention is a pharmaceutical composition, food or food supplement comprising an effective amount of a water-soluble cellulose derivative for regulating, particularly reducing, Stearoyl-CoA Desaturase-1 gene expression in tissues of an animal.

Yet another aspect of the present invention is a pharmaceutical composition, food or food supplement comprising an effective amount of a water-soluble cellulose derivative for preventing or treating a disease of an organ of an animal caused or facilitated by Stearoyl-CoA Desaturase-1 gene expression, particularly Stearoyl-CoA Desaturase-1 gene over-expression.

Yet another aspect of the present invention is a pharmaceutical composition, food or food supplement comprising an effective amount of a water-soluble cellulose derivative for regulating, particularly reducing, ATPAF1 gene expression in tissues of an animal.

Yet another aspect of the present invention is a pharmaceutical composition, food or food supplement comprising an effective amount of a water-soluble cellulose derivative for preventing or reducing oxidative stress or oxidative cell injury in tissues of an animal.

Yet another aspect of the present invention is a pharmaceutical composition, food or food supplement comprising an effective amount of a water-soluble cellulose derivative for preventing or treating a disease of an organ of an animal caused or facilitated by oxidative stress or oxidative cell injury in said organ.

Yet another aspect of the present invention is a method of manufacturing a pharmaceutical composition, food or food supplement for regulating the expression of a gene related to fat metabolism of tissues of an animal, which method comprises combining a water-soluble cellulose derivative with a pharmaceutically acceptable carrier or a food ingredient.

Yet another aspect of the present invention is a method of manufacturing a pharmaceutical composition, food or food supplement for regulating, particularly reducing, Stearoyl-CoA Desaturase-1 gene expression in tissues of an animal, which method comprises combining a water-soluble cellulose derivative with a pharmaceutically acceptable carrier or a food ingredient.

Yet another aspect of the present invention is a method of manufacturing a pharmaceutical composition, food or food supplement for preventing or treating a disease of an organ of an animal caused or facilitated by Stearoyl-CoA Desaturase-1 gene expression, particularly Stearoyl-CoA Desaturase-1 gene over-expression, in tissues of an animal, which method comprises combining a water-soluble cellulose derivative with a pharmaceutically acceptable carrier or a food ingredient.

Yet another aspect of the present invention is a method of manufacturing a pharmaceutical composition, food or food supplement for regulating, particularly reducing, ATPAF1 gene expression in tissues of an animal, which method comprises combining a water-soluble cellulose derivative with a pharmaceutically acceptable carrier or a food ingredient.

Yet another aspect of the present invention is a method of manufacturing a pharmaceutical composition, food or food supplement for preventing or reducing oxidative stress or oxidative cell injury in tissues of an animal, which method comprises combining a water-soluble cellulose derivative with a pharmaceutically acceptable carrier or a food ingredient.

Yet another aspect of the present invention is a method of manufacturing a pharmaceutical composition, food or food supplement for preventing or treating a disease of an organ of an animal caused or facilitated by oxidative stress or oxidative cell injury in said organ, which method comprises combining a water-soluble cellulose derivative with a pharmaceutically acceptable carrier or a food ingredient.

### BRIEF DESCRIPTION OF THE DRAWINGS

FIG. 1 is a schematic representation of a microarray experiment conducted for studying the effect of the method of the present invention on the gene expression in hamster livers.

FIG. 2 is an illustration on the effect of the method of the present invention on gene expression in hamster livers.

### DETAILED DESCRIPTION OF THE PRESENT INVENTION

Water-soluble cellulose derivatives have been suggested for a variety of uses. U.S. Pat. Nos. 5,585,366 and 5,721,221 disclose methods of reducing cholesterol levels in mammalian blood by administering high viscosity water-soluble cellulose derivatives such as hydroxypropyl methylcellulose. U.S. Pat. No. 6,899,892 discloses the use of viscous water-soluble polysaccharides, such as hydroxypropyl methylcellulose, to reduce the percentage of body fat and the level of leptin in the plasma of mammals. U.S. Pat. No. 6,899,892 indicates that lower leptin concentrations are correlated with lower body fat weight. U.S. Pat. No. 5,576,306 discloses that high-viscosity grade cellulose ethers are useful for the reduction of serum lipid levels, particularly total serum cholesterol, serum triglycerides, and low-density lipoprotein (LDL) and/or to attenuate the postprandial rise in blood glucose levels in animals. The above-mentioned effects of the water-soluble polysaccharides, such as cellulose derivatives and other dietary fibers, have been attributed to the viscosity of the dietary fibers. U.S. Pat. No. 6,899,892 specifically discloses that it is the viscosity of the water-soluble fraction of the intestinal contents that is most important. Viscosity-based effects are a kinetic factor since a viscous environment is known to slow down the entire metabolism in the gastrointestinal tract.

Applicants have surprisingly found that administration of a water-soluble cellulose derivative is also useful for regulating the expression of one or more genes related to fat metabolism of tissues of an animal, particularly for regulating the expression of one or more genes for the conversion of saturated fatty acids to monounsaturated fatty acids and/or for regulating the expression of one or more genes related to mitochondrial oxidation pathways, and in particular for regulating, particularly reducing, Stearoyl-CoA Desaturase-1 (SCD1) gene expression and/or ATPAF1 gene expression in tissues, particularly in non-adipose tissues, such as the liver, pancreas, lungs, kidneys, brain, stomach or in muscles. Applicants have found that the water-soluble cellulose derivatives do not only have viscosity-related kinetic effects but also influence genes responsible for saturated fat desaturation and mitochondrial oxidation pathways. Without wanting to be bound to the theory, Applicants believe that the hydrophobic residue of the water-soluble cellulose derivatives contributes to the regulation and normalization of the fat metabolism by water-soluble cellulose derivatives.

Since SCD1 catalyzes the conversion of saturated fatty acids, particularly palmitic acid and stearic acid, to monounsaturated fatty acids, particularly palmitoleate and oleate, Applicants conclude that elevated levels of SCD1 expression, herein designated as SCD1 gene over-expression, in tissues particularly in non-adipose tissues, are an indication of an elevated concentration of saturated fatty acids in these tissues. By the term “gene over-expression” as used herein is meant the expression of a gene which is higher than the normal expression of the gene in healthy animals. For example, obesity is typically accompanied by SCD1 gene over-expression, i.e. by a higher level of SCD1 gene expression than in animals of normal weight.

Furthermore, Applicants conclude that elevated levels of SCD1 gene expression in non-adipose tissues is an indication of oxidative stress in cells or even oxidative cell injury in these tissues. While the adipocytes in adipose tissue have a unique capacity to store excess fatty acids in the form of triglycerides in lipid droplets, non-adipose tissues, such as peripheral tissues, have a limited capacity for storage of lipids. Laura L. Listenberger et al., PNAS, Mar. 18, 2003, vol. 100, no. 6, 3077-3082, “Triglyceride accumulation protects against fatty acid-induced lipotoxicity”, suggests that accumulation of excess lipid in non-adipose tissues leads to cell disfunction and/or cell death, a phenomenon known as lipotoxicity. These authors suggest that lipotoxicity from accumulation of long chain fatty acids is specific for saturated fatty acids and that this selectivity for saturated fatty acids has been attributed to signaling molecules in response to saturated but not unsaturated fatty acids, including reactive oxygen species generation (ROS).

Applicants have found that animals fed with the same fat and caloric diet as control animals show a significantly lower SCD1 gene expression in tissues, particularly in non-adipose tissues, when the diet is supplemented with a water-soluble cellulose derivative. The lower SCD1 expression is an indication that administering a water-soluble cellulose derivative is useful for preventing or reducing oxidative stress or oxidative cell injury in tissues, particularly in non-adipose tissues. Without wanting to be bound to the theory, Applicants conclude from the lower level of SCD1 expression that the concentration of saturated fats is not high enough to induce SCD1 expression, although the animals ingest the same amount of fat as the control animals. Applicants conclude that the lower level of SCD1 expression in such tissues of animals, whose diet is supplemented with a water-soluble cellulose derivative, is sufficient to convert saturated fats into unsaturated fats and into triglyceride storage. The observed lower SCD1 expression in non-adipose tissues of animals, whose diet is supplemented with a water-soluble cellulose derivative but who ingest the same amount of fat as control animals, leads the Applicants to conclude that water-soluble cellulose derivatives prevent or reduce accumulation of excess saturated fats in non-adipose tissues and therefore are useful for preventing or reducing oxidative stress or oxidative cell injury in such tissues which could ultimately lead to cell disfunction and/or cell death.

Applicants have surprisingly found that administration of a water-soluble cellulose derivative is also useful for regulating, particularly reducing, ATPAF1 gene expression in tissues, particularly in non-adipose tissues, of an animal. Based on the findings described in more detail above, Applicants conclude that regulating SCD1 and/or ATPAF1 gene expression contributes to the prevention or reduction of oxidative stress or oxidative cell injury in tissues of an animal, and accordingly to the prevention or treatment of a disease of an organ of an animal caused or facilitated by oxidative stress or oxidative cell injury of said organ. The present invention is particularly useful for the prevention or reduction of oxidative stress or oxidative cell injury and the diseases related thereto which is induced by fat in nutrition, particularly by an imbalanced nutrition with a high fat content.

The term “animal” relates to any animals including human beings. Preferred animals are mammals. The term “mammal” refers to any animal classified as a mammal, including human beings, domestic and farm animals, such as cows, nonhuman primates, zoo animals, sports animals, such as horses, or pet animals, such as dogs and cats.

The cellulose derivatives which are useful in the present invention are water-soluble. The term “cellulose derivative” does not include unmodified cellulose itself which tends to be water-insoluble. The term “water-soluble” as used herein means that the cellulose derivative has a solubility in water of at least 2 grams, preferably at least 3 grams, more preferably at least 5 grams in 100 grams of distilled water at 25° C. and 1 atmosphere.

Preferred cellulose derivatives are water-soluble cellulose esters and cellulose ethers. Preferred cellulose ethers are water-soluble carboxy-C₁-C₃-alkyl celluloses, such as carboxymethyl celluloses; water-soluble carboxy-C₁-C₃-alkyl hydroxy-C₁-C₃-alkyl celluloses, such as carboxymethyl hydroxyethyl celluloses; water-soluble C₁-C₃-alkyl celluloses, such as methylcelluloses; water-soluble C₁-C₃-alkyl hydroxy-C₁₋₃-alkyl celluloses, such as hydroxyethyl methylcelluloses, hydroxypropyl methylcelluloses or ethyl hydroxyethyl celluloses; water-soluble hydroxy-C₁₋₃-alkyl celluloses, such as hydroxyethyl celluloses or hydroxypropyl celluloses; water-soluble mixed hydroxy-C₁-C₃-alkyl celluloses, such as hydroxyethyl hydroxypropyl celluloses, water-soluble mixed C₁-C₃-alkyl celluloses, such as methyl ethyl celluloses, or water-soluble alkoxy hydroxyethyl hydroxypropyl celluloses, the alkoxy group being straight-chain or branched and containing 2 to 8 carbon atoms. The more preferred cellulose ethers are methylcellulose, methyl ethyl cellulose, hydroxyethyl cellulose, hydroxypropyl cellulose, hydroxyethyl methylcellulose, hydroxypropyl methylcellulose, and carboxymethyl cellulose, which are classified as water-soluble cellulose ethers by the skilled artisans. The most preferred water-soluble cellulose ethers are methylcelluloses with a methyl molar substitution DS_{methoxyl} of from 0.5 to 3.0, preferably from 1 to 2.5, and hydroxypropyl methylcelluloses with a DS_{methoxyl} of from 0.9 to 2.2, preferably from 1.1 to 2.0, and a MS_{hydroxypropoxyl} of from 0.02 to 2.0, preferably from 0.1 to 1.2. The methoxyl content of methyl cellulose can be determined according to ASTM method D 1347-72 (reapproved 1995). The methoxyl and hydroxypropoxyl content of hydroxypropyl methyl cellulose can be determined by ASTM method D-2363-79 (reapproved 1989). Methyl celluloses and hydroxypropyl methylcelluloses, such as K100M, K4M, K1M, F220M, F4M and J4M hydroxypropyl methylcellulose are commercially available from The Dow Chemical Company).

The water-soluble cellulose derivative generally has a viscosity of from 5 to 2,000,000 cps (=mPa·s), preferably from 50 cps to 200,000 cps, more preferably from 75 to 100,000 cps, in particular from 1,000 to 50,000 cps, measured as a two weight percent aqueous solution at 20 degrees Celsius. The viscosity can be measured in a rotational viscometer.

The cellulose derivative can be administered or consumed in or as a pharmaceutical composition, food or food supplement. The term “food” as used herein includes solid food as well as liquid food like beverages. The desired time period of administering the water-soluble cellulose derivative can vary depending on the amount of water-soluble cellulose derivative consumed, the general health of the animal, the level of activity of the animal and related factors. Since the present invention is particularly useful for the prevention or reduction of oxidative stress or oxidative cell injury and the diseases related thereto which are induced by an imbalanced nutrition with a high fat content, it may be advisable to administer or consume the water-soluble cellulose derivative as long as nutrition with a high fat content is consumed. Generally administration of at least 1 to 12 weeks, preferably 3 to 8 weeks is recommended to reach a lower SCD1 and/or ATPF1 gene expression and, accordingly, a prevention or reduction of oxidative stress or oxidative cell injury, as compared to animals consuming the same diet in the absence of a water-soluble cellulose derivative. The water-soluble cellulose derivatives are generally considered to be non-toxic and non-nutritive, are generally not absorbed by the gastrointestinal tract, and generally do not contain allergenic materials. It is to be understood that the duration and daily dosages of administration as disclosed herein are general ranges and may vary depending on various factors, such as the specific cellulose derivative, the weight, age and health condition of the animal, and the like. It is advisable to follow the prescriptions or advice of medical doctors or nutrition specialists when consuming the cellulose derivatives.

Although water-soluble cellulose derivatives have been used in a variety of foodstuffs to improve certain functional properties, such as emulsification, texture or moisture retention, the amounts used are usually less than 0.5% of the foodstuff. These levels are generally too low to have a significant effect on SCD1 and/or ATPF1 gene expression and, accordingly, on prevention or reduction of oxidative stress or oxidative cell injury and the diseases related thereto.

In contrast to the small amounts employed in commercially available compositions and processed foodstuffs, according to the present invention the water-soluble cellulose derivatives are preferably used for preparing food or a food supplement which comprises from 0.5 to 20 weight percent, preferably from 2 to 15 weight percent, more preferably from 4 to 12 weight percentage of one or more water-soluble cellulose derivatives. The given weight percentages relate to the total amount of the water-soluble cellulose derivatives. The amount administered is preferably in the range of from 1 to 10 percent of the total daily weight of the diet of the mammal on a dry weight basis. Preferably, the water-soluble cellulose derivatives are administered or consumed in sufficient amounts throughout the day, rather than in a single dose or amount.

Although the water-soluble cellulose derivatives are preferably administered in combination with food or as foodstuff, alternatively they can be administered in solution or in powder form or as pharmaceutical compositions. Pharmaceutical compositions containing water-soluble cellulose derivatives can be administered with a pharmaceutically acceptable carrier in a pharmaceutical unit dosage form. Pharmaceutically acceptable carriers include tableting excipients, gelatin capsules, or carriers such as a polyethylene glycol, a natural gel, and the like. Pharmaceutical unit dosage forms include tablets, capsules, gelatin capsules, pre-measured powders, pre-measured solutions, and the like. Hence, the water-soluble cellulose derivatives may be formulated as tablets, granules, capsules, suspensions and the like.

Regardless whether the water-soluble cellulose derivative is administered in solution or powder form, as a pharmaceutical composition or is combined with other food ingredients, the amount of administered water-soluble cellulose derivative is generally in the range of from 10 to 300 milligrams of water-soluble cellulose derivative per pound of mammal body weight per day. About 2 g to about 30 g, preferably about 3 g to about 15 g of water-soluble cellulose derivative are ingested daily by a large mammal such as a human.

While the method of administration or consumption may vary, the water-soluble cellulose derivatives are preferably ingested by a human as an food ingredient of his or her daily diet. The water-soluble cellulose derivatives can be combined with a liquid vehicle, such as water, milk, vegetable oil, juice and the like, or with an ingestible solid or semi-solid foodstuff, such as “veggie” burgers or bakery products. A number of foodstuffs are generally compatible with water-soluble cellulose derivatives. Examples of such foodstuffs are disclosed by M. K. Weibel et al., U.S. Pat. No. 4,923,981, the disclosure of which is incorporated by reference herein. For example, a water-soluble cellulose derivative may be mixed into foods such as milk shakes, milk shake mixes, breakfast drinks, juices, flavored drinks, flavored drink mixes, yogurts, puddings, ice creams, ice milks, frostings, frozen yogurts, cheesecake fillings, candy bars, including “health bars” such as granola and fruit bars, gums, hard candy, mayonnaise, pastry fillings such as fruit fillings or cream fillings, cereals, breads, stuffing, dressings and instant potato mixes. An effective amount of water-soluble cellulose derivatives can also be used as a fat-substitute or fat-supplement in salad dressings, frostings, margarines, soups, sauces, gravies, mayonnaises, mustards and other spreads. Therefore, “food ingredients,” as the term is used herein, includes those ingredients commonly employed in recipes for the above foodstuffs, including flour, oatmeal, fruits, milk, eggs, starch, soy protein, sugar, sugar syrups, vegetable oils, butter, emulsifying agents such as lecithin, and the like.

The water-soluble cellulose derivatives can be partially or fully hydrated before they are orally ingested. For example, the water-soluble cellulose derivatives may be dispersed in a sufficient amount of water, milk, juice, flavored water, hot chocolate, soy milk, cream, or other liquid to make a drink item that can be consumed to administer an effective amount of the water-soluble cellulose derivatives. The water-soluble cellulose derivative may be dispersed in a sufficient amount of water to make a syrupy liquid that is then mixed with one or more food ingredients such as flours, oatmeal, cornmeal, rice, barley, wheat germ, and other cereal products to made a paste or dough, the latter being subsequently treated to create an appealing foodstuff by procedures such as baking, extruding, and the like, to provide edible foodstuffs. Colorings and flavorings may be added as may be appropriate to add to the attractiveness of the foodstuff.

Since the present invention is also useful for preventing or reducing oxidative stress or oxidative cell injury, particularly oxidative stress or oxidative cell injury induced by fat in nutrition, the present invention is also useful for preventing or treating a disease that is caused or facilitated by oxidative stress or oxidative cell injury of said organ. Such diseases are numerous. For example, the present invention is useful for preventing or treating liver diseases, such as hepatitis; mitochondrial and/or metabolic diseases, such as insulin resistance, Type II Diabetes, or hypercholesterolemia and/or hypertension related to diabetes, atherosclerosis; ischemic injuries, such as cardiac ischemic injury; inflammatory diseases, such as inflammatory bowel disease; cardiovascular disorders, such as coronary heart disease or post-ischemic arrhythmias; neurological diseases, such as Alzheimer's, stroke, bovine Spongiform Encephalopathy (BSE; Mad Cow Disease); Creutzfeld Jacob Disease (CJD; human variant of BSE); muscle damage; or for the treatment of AIDS.

The present invention is particularly useful for preventing or treating diseases that are associated by the skilled persons with the expression, particularly over-expression of Stearoyl-CoA Desaturase-1 in tissues of animals, including mitochondrial and/or metabolic diseases, such as insulin resistance, Type II Diabetes or hypercholesterolemia and/or hypertension related to diabetes.

The present invention is further illustrated by the following examples which are not to be construed to limit the scope of the invention. Unless otherwise mentioned, all parts and percentages are by weight.

### EXAMPLES

The effect of administering a hydroxypropyl methylcellulose (HPMC) to hamsters was tested. The HPMC had a methoxyl content of 19-24 percent, a hydroxypropoxyl content of 7-12 percent and a viscosity of about 100,000 mPa·s, measured as a 2 wt. % aqueous solution at 20° C. and is commercially available from The Dow Chemical Company under the Trademark METHOCEL K100M hypromellose.

An animal study was done with 9 male golden Syrian hamsters with a starting body weight of 70-90 grams (Sasco strain, Charles River, Wilmington, Mass.) in each of the three diets specified below. The animal study was approved by the Animal Care and Use Committee, Western Regional Research Center, USDA, Albany, Calif.

The hamsters were placed on either a low fat (LF), high fat (HF), or high fat diet containing hydroxypropyl methylcellulose (HF+HPMC) for four weeks.

The LF diet contained 8 percent microcrystalline cellulose, 4 percent butter, 2 percent corn, 20 percent protein, 61 percent starch and 5 percent other materials consisting of vitamins, minerals, choline bitartrate and methionine. The average weight of these hamsters after the LF diet was 126±2 g.

The HF diet contained 7 percent microcrystalline cellulose, 14 percent butter, 5 percent corn, 19 percent protein, 50 percent starch and 5 percent other materials consisting of vitamins, minerals, choline bitartrate and methionine. The HF diet contained about 35 percent fat calories, which corresponds to the fat calories in an American diet. In the years 1984-1995 in the U.S the mean fat intake of males with 19-50 years of age was 33-37%, based on the total calories. The average weight of these hamsters after the HF diet was 130±3 g.

The HF+HPMC diet was the same as the HF diet, except that HPMC was substituted for the microcrystalline cellulose. The average weight of these hamsters after the HF+HPMC diet was 122±2 g.

The insulin resistance of the animals was determined by the Euglycemic Hyperinsulinemic Clamp Method, as described by DeFronzo et al, 1979. A primed-constant intravenous infusion of human biosynthetic insulin was administered as a 180 microgram/kg bolus followed by 18 microgram/kg/min for 2 h. The human biosynthetic insulin was commercially available from Eli Lilly, Indianapolis, Ind. under the trademark Humulin R. The blood glucose level was maintained at baseline level by infusing a 25 percent aqueous solution of glucose by syringe pump while periodically monitoring blood glucose using a Fast Glucose Meter-Precision Xtra, MediSense, Bedford, Mass., USA every 10 minutes. Blood samples of 0.25 ml each were drawn at 100, 110 and 120 minutes to assess the steady state glucose and insulin levels. At the end of the Euglycemic Hyperinsulinemic Clamp Method, animals were exsanguinated by cardiac puncture under isofluorane anesthesia. The blood samples were collected by syringes containing a small amount of EDTA (ethylenediaminetetra acetic acid) and then centrifuged at 3,000 rpm for 20 min. Plasma was separated and stored at −80° C. until use. Livers were excised, blotted dry and weighed. Part of the liver was preserved in a 2.5 percent of glutaraldehyde in a phosphate buffered saline solution (PBS) for electron microscopy. Adipose tissues were taken from the retroperitoneal and femoral fat pad, weighed and stored at −80° C.

The hamsters fed with the HF diet showed insulin resistance, whereas the hamsters fed with the LF diet and the hamsters fed with the HF+HPMC diet showed normal insulin sensitivity.

A Microarray study was done as follows:

Livers from 3 hamsters in each of the two treatment groups HF and HF+HPMC were compared. The following animal pairs were studied in the microarray study:

| | | | HF − HF + HPMC |
|---|---|---|---|
| | High Fat diet | High Fat + HPMC diet | Animal Pairs |
| Animal | A | D | A/D |
| number | B | E | B/E |
| | C | F | C/F |

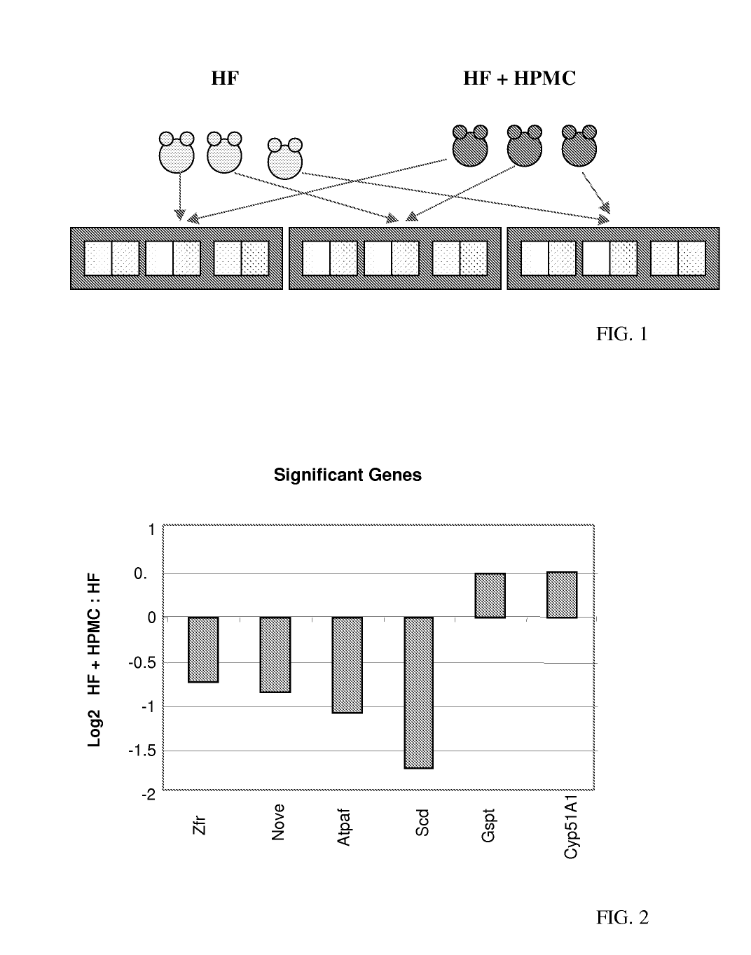

Pairs of animals were assigned to 3 separate microarray chips. Each chip contained about 7,000 distinct genes. Two diet treatments were compared per chip. Messenger ribonucleic acid (RNA) was extracted from each liver and applied to the chips. A diagram of the experiment is shown in FIG. 1. The arrows in FIG. 1 mean:
The gene expression of animal A (HF diet) was compared with the gene expression of animal D (HF+HPMC diet); the gene expression of animal B (HF diet) was compared with the gene expression of animal E (HF+HPMC diet); and the gene expression of animal C(HF diet) was compared with the gene expression of animal F (HF+HPMC diet); The first selection criteria was that the ratio of gene expression be at least 1.4 different and that p<0.1 within a single chip. A second criteria was that at least two chips agree with each other.

Results of the HF+HPMC diet in comparison with the HF diet:

About 250 differences in expressed genes resulted which fit the first selection criteria but only six difference in expressed genes remained after the second criteria was imposed in the comparison between the dietary induced insulin resistant animals, which had been fed with the HF diet, and animals of normal insulin sensitivity, which had been fed with the HF+HPMC diet.

| Gene | Log 2 HF + HPMC:HF Ratio | HF + HPMC:HF Ratio |
|---|---|---|
| Scd1 | −1.7 | 0.31 |
| Atpaf1 | −1.1 | 0.47 |
| Cyp51A1 | 0.62 | 1.54 |

Of these 6 differences in gene expression the most prominent was Scd1, Stearoyl Co-A Desaturase-1 (SCD1). Although fed with the identical high fat diet, the hamsters that were additionally fed with HPMC (instead of microcrystalline cellulose) had a significantly lower level of Scd1. The second most prominently expressed gene was Atpf1, ATP synthase mitochondrial F1 complex assembly factor 1. Mitochondria are the organelles that oxidize fat for energy. The presence of higher levels of this assembly factor in animals fed with the HF diet than in animals fed with the HF+HPMC diet is evidence for a higher level of fat oxidation for energy production in the animals fed with the HF diet. Cyp51A1 is a cytochrome P450 enzyme that is necessary cholesterol synthesis. It's higher level in the HF+HPMC fed animals may be due to the increased excretion of bile acids in HPMC fed hamsters. Cholesterol is a precursor of bile acids. Fecal excretion of bile acids generally requires hepatic synthesis of cholesterol.

1. A method of regulating the expression of a gene related to fat metabolism of tissues of an animal, which method comprises the step of administering to the animal an effective amount of a water-soluble cellulose derivative.

2. The method of claim 1 wherein the expression of a gene related to the fat metabolism of non-adipose tissues is regulated.

3. The method of claim 1 wherein the expression of a gene for the conversion of saturated fatty acids to monounsaturated fatty acids is regulated.

4-5. (canceled)

6. The method of claim 1 wherein the animal is a mammal.

7. The method of claim 1 wherein the cellulose derivative is a water-soluble C₁-C₃-alkyl cellulose, a water-soluble C₁-C₃-alkyl hydroxy-C₁₋₃-alkyl cellulose, a water-soluble hydroxy-C₁₋₃-alkyl cellulose, a water-soluble mixed hydroxy-C₁-C₃-alkyl cellulose, or a water-soluble mixed C₁-C₃-alkyl cellulose.

8. The method of claim 1 wherein from 10 to 300 milligrams of water-soluble cellulose derivative per pound of mammal body weight is administered per day in the form of a pharmaceutical composition, food or food supplement.

9. A method of regulating Stearoyl-CoA Desaturase-1 gene expression in tissues of an animal, which method comprises the step of administering to the animal an effective amount of a water-soluble cellulose derivative.

10. The method of claim 9 wherein Stearoyl-CoA Desaturase-1 gene expression in non-adipose tissues of an animal is reduced.

11. The method of claim 9 wherein Stearoyl-CoA Desaturase-1 gene expression in non-adipose tissues of a mammal is reduced.

12. The method of claim 11 wherein Stearoyl-CoA Desaturase-1 gene in the liver, pancreas, lungs, kidneys, brain, stomach or in muscles of a mammal is reduced.

13. The method of claim 9 wherein the cellulose derivative is a water-soluble C₁-C₃-alkyl cellulose, a water-soluble C₁-C₃-alkyl hydroxy-C₁₋₃-alkyl cellulose, a water-soluble hydroxy-C₁₋₃-alkyl cellulose, a water-soluble mixed hydroxy-C₁-C₃-alkyl cellulose, or a water-soluble mixed C₁-C₃-alkyl cellulose.

14. The method of claim 9 wherein from 10 to 300 milligrams of water-soluble cellulose derivative per pound of mammal body weight is administered per day in the form of a pharmaceutical composition, food or food supplement.

15-19. (canceled)

20. A method of preventing or treating a disease of an organ of an animal caused or facilitated by Stearoyl-CoA Desaturase-1 gene expression, which method comprises the step of administering to the animal an effective amount of a water-soluble cellulose derivative.

21. The method of claim 20 wherein mitochondrial or metabolic diseases are prevented or treated.

22. The method of claim 20 wherein said animal is a human being.

23-41. (canceled)
